(19) United States
(12) Patent Application Publication
(10) Pub. No.: US 2026/0265489 A1
(43) Pub. Date: Sep. 10, 2026

(54) DISCOID LIQUID CRYSTAL MOLECULE FUNCTIONAL MODIFIER, PREPARATION METHOD THEREFOR AND USE THEREOF

(71) Applicant: WUHAN UNIVERSITY OF TECHNOLOGY, Wuhan (CN)

(72) Inventors: Lijie DONG, Wuhan (CN); Meng PAN, Wuhan (CN); Yang ZHANG, Wuhan (CN); Xiaoyu LI, Wuhan (CN); Guanghui ZHAO, Wuhan (CN); Xiaodong ZHU, Wuhan (CN)

(21) Appl. No.: 18/871,812

(22) PCT Filed: Mar. 8, 2023
(86) PCT No.: PCT/CN2023/080296

§ 371 Date: Dec. 5, 2024

(30) Foreign Application Priority Data
Oct. 19, 2022 (CN) 202211279179.X

(51) Int. Cl. C08K 5/20; C07C 233/88; C08J 3/20; C08J 3/215; C08J 5/18

(52) U.S. Cl. CPC C08K 5/20; C07C 233/88; C08J 3/201; C08J 3/215; C08J 5/18; C08J 2323/12; C08J 2327/16

(57) ABSTRACT

Provided are a discoid liquid crystal molecule functional modifier, a preparation method therefor and use thereof. A discoid liquid crystal molecule functional modifier is first synthesized by using a trimesoyl chloride monomer and a flexible carbon chain compound with an amino group as raw materials; and then the prepared discoid liquid crystal molecule functional modifier is mixed with a polymer matrix, and a composite film material with good performance is prepared by means of a tape casting method or a melt extrusion method. By using the discoid liquid crystal molecule functional modifier, the crystalline structure of a polymer is effectively adjusted and controlled, the grain size of the polymer is reduced, the content of a β phase is increased, and an interface interaction between the discoid liquid crystal molecule functional modifier and the polymer matrix is increased, such that the dielectric constant, the breakdown field strength, the mechanical properties and the energy storage density of the composite film material are improved. The discoid liquid crystal molecule functional modifier has the advantages of readily available raw materials, simple synthesis method, adjustable and controllable size, high yield, less dosage, easy purification and the like, is suitable for industrial production, and has good application prospect in the aspects of adjusting and controlling the crystalline structure and performance of the polymer film material.

### TECHNICAL FIELD

The present disclosure relates to the technical field of polymer film materials and processing aids, and in particular to a discoid liquid crystal molecule functional modifier, a preparation method therefor and use thereof.

### BACKGROUND

A polymer film material has excellent comprehensive performances, including mechanical properties, electrical properties, heat resistance, chemical resistance, etc., as well as low density, easy processing, transparency, folding resistance and relatively low product manufacturing cost. These advantages make the polymer film material play a more and more important role in various fields of science and technology as well as national economy. The polymer film material can also be flexibly compounded with various different functional materials to form a complicated material system having excellent characteristics, thereby further exerting the advantages of functional fillers and avoiding the limitations of a single material. With the development of a microelectronic technology, the polymer film material simultaneously having excellent mechanical properties and electrical properties is the key to achieving miniaturization of devices and systems.

Crystalline performances have important effects on the mechanical properties, optical property, electrical properties and thermal property of the polymer. The arrangement mode of molecular chains in the polymer affects the formation of a crystal core, the growth of crystal grains and the crystallization perfection degree of the polymer. The modulus, rigidity, hardness and the like of the polymer increase with the increase in crystallinity, and creep resistance and stress relaxation can also be improved with the increase in crystallinity. In general, the increased crystallinity indicates reduced impact strength, elongation at break and other properties of the polymer. However, when the size of a ball crystal is reduced, that is, when the crystal size decreases and the main crystal form is a β crystal form, and the impact performance of the polymer is slightly improved. In addition, the crystalline performances also has a significant impact on the optical properties of the polymer, mainly affecting transparency. This is because crystallization increases refractive index difference between crystalline and amorphous regions, making it easier for refraction and reflection to occur at the interface, thereby resulting in a decrease in transmittance. Thus, a majority of amorphous polymers are transparent, such as polypropylene (PP), which belongs to semi-transparent polymers, and its transparency can be controlled by crystallization. The addition of a nucleating agent into PP can significantly reduce the crystallinity and ball crystal size to obtain a PP product containing small crystals, thereby improving the transparency. The crystallinity and the content of a polar crystal phase also greatly affect the electrical properties of the polymer. For example, polyvinyliene fluoride (PVDF) undergoes α-β phase transition and orientation, which can enhance dielectric polarization; meanwhile, the orientation causes an increase in Young's modulus of PVDF, thereby improving the breakdown strength and the energy storage density. Perfect crystallization can also increase a thermal deformation temperature, thereby increasing the usage temperature of the polymer.

The commonly used methods for adjusting and controlling the crystalline performances of the polymer include control of a molding temperature, change in production processes (for example, using electrospinning), stretching (strain-induced crystallization), heat treatment, addition of a nucleating agent, etc. Among them, the control of the molding temperature, stretching, heat treatment and other methods often have complex processes and high energy consumption, so electrospinning is more affected by process conditions, and huge energy consumption increases production costs, leading to difficult achievement of industrial production in large batch. In addition, the stretching, heat treatment and other methods are limited by sites and equipment when large-area films and devices are processed, which easily leads to inhomogeneous temperature, thereby affecting the homogeneity and yield of a product, and there is also a problem of increased costs due to high energy consumption. Based on a polymer heteronucleation principle, the addition of a nucleating agent and other fillers is the most convenient and effective key method to adjust and control the crystalline performances of the polymer film. The addition of an appropriate nucleating agent during the production can effectively adjust and control the crystalline performances of the polymer, such as improvement of crystal grain size and increase in β phase content on the basis that energy consumption does not additionally increase, thereby improving other properties of the polymer.

The addition of an appropriate functional filler to adjust and control the content of a polar phase crystal form and crystallinity of the polymer is also an effective way to improve dielectric constant, breakdown field strength and other electrical properties of the polymer. However, agglomeration, defects and other problems brought by the high addition amount of the filler significantly reduce the breakdown field strength of a polymer composite material, and reduce flexibility and transparency to a certain extent. In addition, at present, a majority of high-performance nanofillers are low in synthesis yield, surface modification treatment makes the preparation process of the fillers complex and cost-effective, and may also sacrifice the mechanical properties of a dielectric film, so that the film processing technology becomes quite complex and is difficult to industrialize.

To sum up, it is urgent to develop a novel industrially producible functional modifier that can effectively adjust and control the crystalline performances of the polymer so as to improve its mechanical properties while promoting electrical properties.

### SUMMARY

In view of the problems existing in the current polymer film functional modifier, such as high addition amount, complex process, difficulty in effectively improving the crystalline performances, a novel functional modifier that is simple, easily available and industrially producible is developed. The novel functional modifier can effectively improve the crystallization rate of the polymer, reduce the sizes of crystal grains and improve the crystallinity and polar phase content on the premise that process steps and energy consumption are not additionally increased, so as to provide a new idea for adjusting and controlling the crystalline structure and mechanical properties of the polymer and improving the electrical properties of the polymer film material.

The first objective of the present disclosure is to provide a discoid liquid crystal molecule functional modifier, the functional modifier having the following molecule structure:

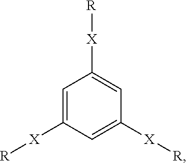

where X is selected from any one of —CONH—, —NHCO—, —O—, —COO—, —OOC—, —C═O— and —OCO—, and R is selected from any one of —(CH₂)ₓCH₃, —(CH₂)ₓCOOH, —(CH₂)ₓOH, —(CH₂)ₓNH₂, —(CH₂)ₓCONH(CH₂)_{Y}CH₃, —(CH₂)ₓNHCO(CH₂)_{Y}CH₃, —(CH₂)ₓCOO(CH₂)_{Y}CH₃, —(CH₂)ₓOOC(CH₂)_{Y}CH₃, —(CH₂)ₓCO(CH₂)_{Y}CH₃, —(CH₂)ₓO(CH₂)_{Y}CH₃ or flexible side chains of the aforementioned groups substituted by a halogen atom, an amino group, a carboxyl group, a hydroxyl group and a sulfo group at any positions, and the values of X and Y are positive integers within 18 (inclusive).

Further, X is specifically—CONH— or —NHCO—, and R is specifically—(CH₂)₅CH₃, —(CH₂)₅COOH, —(CH₂)₆CH₃ or —(CH₂)₆NH₂.

The second objective of the present disclosure is to provide a method for preparing the discoid liquid crystal molecule functional modifier, comprising the following steps: reacting by using a trimesoyl chloride monomer and a flexible carbon chain compound with an amino group as raw materials under the conditions of no water, no oxygen and no more than 5° C., separating and purifying to obtain the discoid liquid crystal molecule functional modifier.

Furthermore, the flexible carbon chain compound with the amino group is specifically organic amine or amino acid, which comprises n-hexamine, n-heptamine, n-octylamine and ω-amino acid.

Further, a molar ratio of the trimesoyl chloride monomer to the flexible carbon chain compound with the amino group required by reaction is 1:2.2-3.8.

Further, the specific process of the reaction is as follows: dissolving the trimesoyl chloride monomer and the flexible carbon chain compound with the amino group into an organic solvent (such as trichlormethane) under the conditions of no water, no oxygen and no more than 5° C. to be uniformly mixed, then performing a thermal insulation reaction for a while followed by heating to room temperature to continue the reaction over a period of time, and then subjecting the reaction product to organic solvent (such as bichloromethane) extraction, water washing, column chromatography separation or recrystallization, rotary evaporation and drying, so as to obtain a target product.

The third objective of the present disclosure is to provide use of the discoid liquid crystal molecule functional modifier in adjusting and controlling a crystalline structure and performances of a polymer film material.

Further, the polymer is selected from fluorinated polar ferroelectric polymers that easily form intermolecular hydrogen bonds or copolymers thereof, including polyvinylidene fluoride (PVDF), polyvinylidene difluoride-hexafluoropropene (PVDF-HFP), a polyvinylidene fluoride-trifluoroethylene copolymer (PVDF-TrFE), a polyvinylidene fluoride-chlorotrifluoroethylene copolymer (PVDF-CTFE), an ethylene-tetrafluoroethylene copolymer (ETFE), an fluorinated ethylene propylene copolymer (FEP), polychlorotrifluoroethylene (PCTFE), a copolymer of perfluoropropyl perfluorovinyl ether and polytetrafluoroethylene (PFA), polyvinyl chloride (PVC), polyhexamethylene adipate (PA66), polyamide (PA6), polyethylene terephthalate (PET), polymethyl methacrylate (PMMA), polypropylene (PP), polyphenylene sulfide (PPS), etc.

Further, the use method of the discoid liquid crystal molecule functional modifier is specifically as follows: (a) respectively dissolving the discoid liquid crystal molecule functional modifier and a polymer into a solvent, then uniformly mixing two solutions and filming; or (b) heating and melting the polymer, then adding the discoid liquid crystal molecule functional modifier, and performing extrusion filming.

Further, the mass fraction of the discoid liquid crystal molecule functional modifier in the polymer film material is 0.0001%-10%. The study shows that different contents of discoid liquid crystal functional modifiers can effectively adjust and control the crystalline performances of the polymer film material so as to obtain polymer film composite materials with different crystalline sizes, crystal forms and crystallinity.

Further, the thickness of the polymer film material is 1 μm-100 μm, preferably 10 μm-25 μm.

Further, in the method (a), the solvents for dissolving the discoid liquid crystal molecule functional modifier and the polymer are specifically non-protonic solvents, which comprise N-methylpyrrolidone, N, N′-dimethylacetamide, N, N′-dimethylformamide and ethanol.

Furthermore, the solvents for dissolving the discoid liquid crystal molecule functional modifier and the polymer are the same or different, preferably the same.

Further, in the method (a), the solid contents of the discoid liquid crystal molecule functional modifier solution and the polymer solution which are obtained by dissolution are 0.001 wt %-50 wt % (preferably 0.1 wt %-5 wt %) and 1 wt %-30 wt % (preferably 6 wt %-15 wt %) respectively, a volume ratio of the two solutions during the mixing is 1:20-20:1, and the filming method is selected from at least one of tape casting, spin coating and pulling methods.

Further, in the method (b), the polymer is first heated and melted, and then a discoid liquid crystal molecule functional modifier solution with a solid content of 0.001 wt %-50 wt % (preferably 0.1 wt %-5 wt %) is prepared according to the method (a), subsequently the prepared discoid liquid crystal molecule functional modifier solution is uniformly sprayed to the surface of the polymer melt, and the obtained mixed material is dried in vacuum and then subjected to extrusion filming in an extruder.

Further, in the method (b), the vacuum drying temperature of the mixed material is 40° C.-100° C., the mixed material is heated in the extruder in four stages, the heating temperatures of the four stages are 120° C.-140° C., 130° C.-150° C., 180° C.-220° C. and 170° C.-210° C. in sequence, and the extruded material is pulled by a puller, and a distance between the puller and the extruder head is controlled at 15-45 cm so as to ensure that the material is sufficiently cooled during the pulling, and the pulling speed of no more than 30 r/min.

Compared with the prior art, the present disclosure has the following improvement effects:
(1) in order to solve the problems in the existing polymer film materials, such as high addition amount, complex production process and difficulty in effectively improving the crystalline performances of the polymer, a bran-new discoid liquid crystal molecule functional modifier has been developed, and the crystalline structure and performances of the polymer are successfully adjusted and controlled by utilizing the discoid liquid crystal molecule functional modifier;
(2) the discoid liquid crystal molecule provided by the present disclosure has the advantages of easily available raw materials, simple synthesis method, adjustable size, high yield, less dosage, easy purification and the like, does not need additional and cumbersome surface chemical modifications, and therefore is suitable for industrial production;
(3) when in use, only a few amount of discoid liquid crystal molecule functional modifier needs to be mixed with the polymer, and the film composite material can be prepared by the tape casting or melt extrusion method, the discoid liquid crystal molecule functional modifier can effectively adjust and control the crystalline structure of the polymer, reduce the grain size of the polymer, increase the content of the β phase, and increase the interface interaction between the discoid liquid crystal molecule functional modifier and the polymer matrix, thereby improving the dielectric constant, breakdown field strength, mechanical properties and energy storage density, which is also proved by a comparative experiment;
(4) the discoid liquid crystal molecule functional modifier provided by the present disclosure avoids the currently common problems that the breakdown field strength and electrical properties are reduced due to uneven dispersion of inorganic materials in polymer matrices, can improve the energy storage density of the film composite materials while maintaining other excellent properties such as inherent flexibility and high breakdown field strength of polymers on the premise that process steps and energy consumption are not additionally increased.

### BRIEF DESCRIPTION OF THE DRAWINGS

FIG. 1 is a 1H-NMR nuclear magnetic resonance spectrum of a discoid liquid crystal small molecule BTA-1 prepared in example 1;

FIG. 2(a) is DSC curves a crystalline performance graph of polymer composite films corresponding with to different content discoid liquid crystal molecule functional modifier contents in example 1;

FIG. 2(b) is FTIR spectra of the nanocomposite film with different content in example 1;

FIG. 2(c) is a crystalline performance graph of polymer composite films with different content in example 1;

FIG. 2(d) is a fraction of the β conformations with different content in example 1;

FIG. 3(a) is a graph showing dielectric constant of polymer composite films with different content in example 1;

FIG. 3(b) is a graph showing dielectric loss of polymer composite films with different content in example 1;

FIG. 3(c) is a graph unipolar D-E loops of polymer composite films with different content in example 1;

FIG. 3(d) is a discharged energy density and ferroelectric testing curves charge-discharge efficiency of polymer composite films corresponding to with different discoid liquid crystal molecule functional modifier contents content in example 1;

FIG. 4 is a graph showing breakdown strengths of polymer composite films with corresponding to different content discoid liquid crystal molecule functional modifier contents in example 1; and

FIG. 5 (a) is a structural diagram of the discoid liquid crystal small molecules BTA-1, as prepared in Example 1; four discoid liquid crystal small molecules prepared in examples 1-4.

FIG. 5(b) is a structural diagram of the discoid liquid crystal small molecules BTA-2, as prepared in Example 2;

FIG. 5(c) is a structural diagram of the discoid liquid crystal small molecules BTA-3, as prepared in examples 1 Example 3; and

FIG. 5(d) is a structural diagram of the discoid liquid crystal small molecules BTA-4, as prepared in Example 4.

### DETAILED DESCRIPTION OF THE EMBODIMENTS

In order to make persons of ordinary skill in the art sufficiently understand the technical solution and beneficial effects of the present disclosure, the present disclosure will be described in detail in combination with specific embodiments and drawings.

### Example 1

### (1) Synthesis of Discoid Liquid Crystal Small Molecule BTA-1

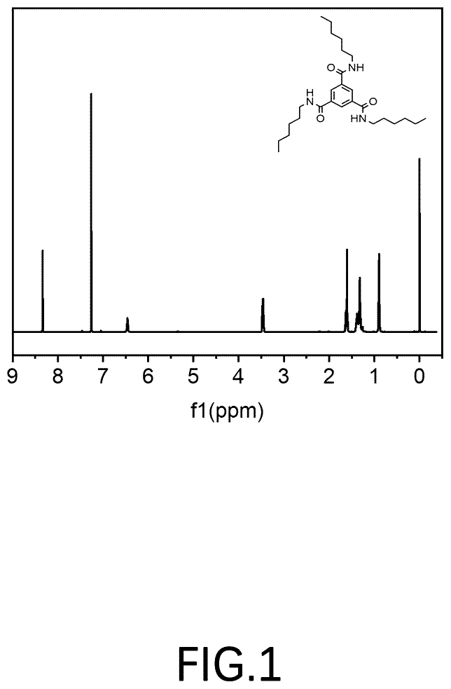

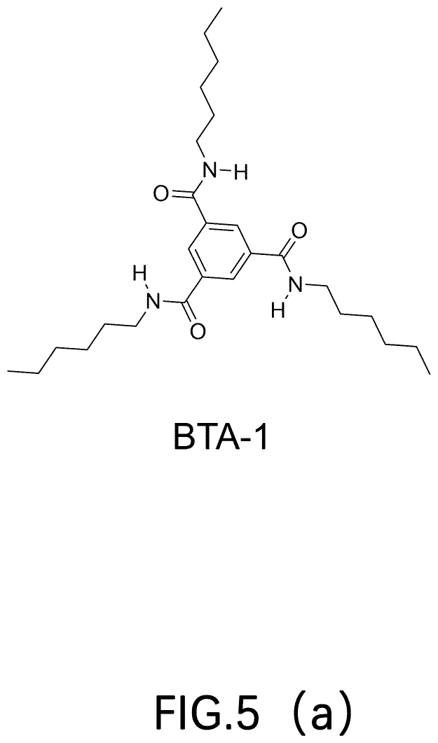

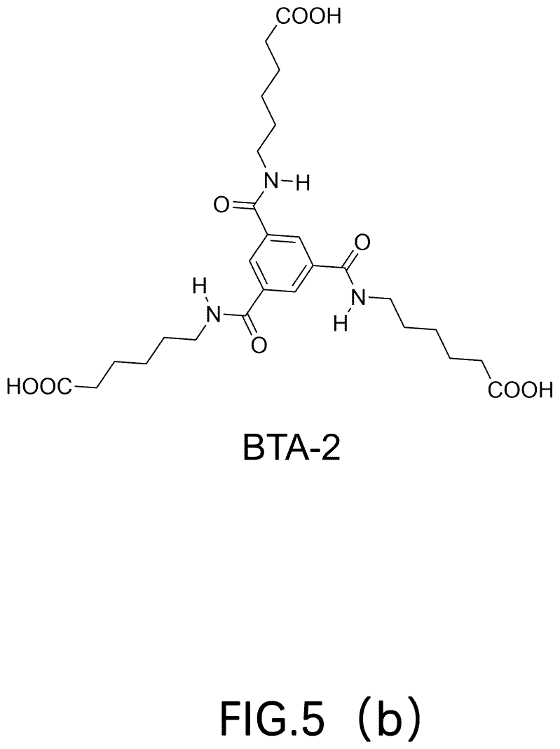

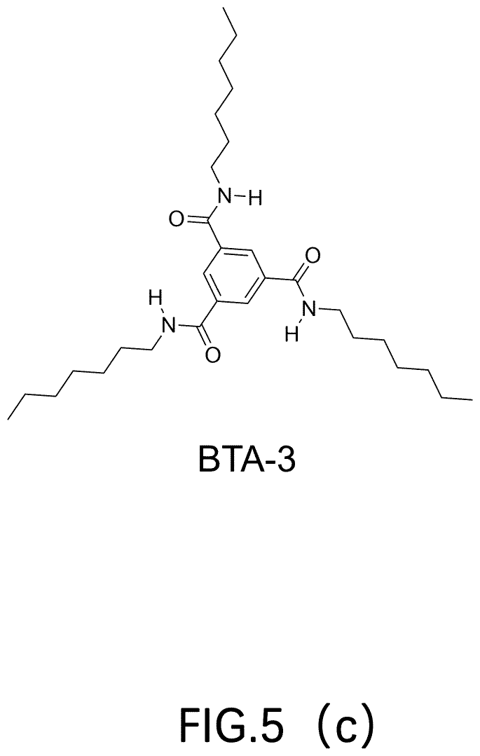

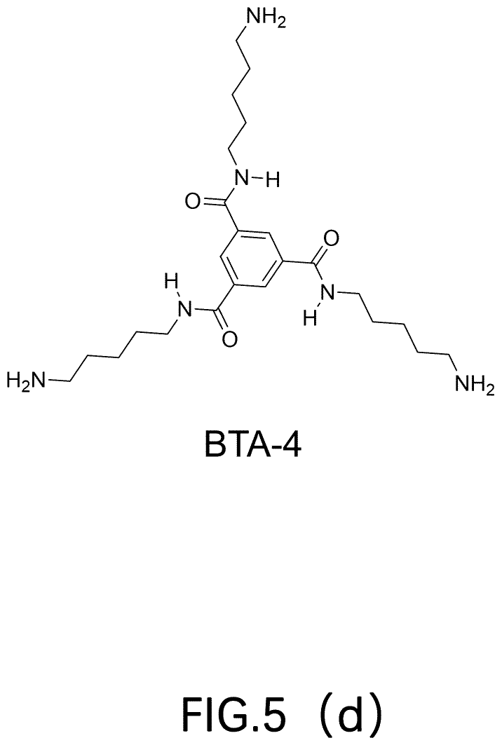

3.76 mmol of 1,3,5-benzoyl chloride and 11.7 mmol of n-hexane were added into 30 mL of trichloromethane in a nitrogen atmosphere under the conditions of no water and oxygen at 0° C. to be uniformly mixed, and then the obtained mixture was subjected to a thermal insulation reaction for 30 min at 0° C. and subsequently continued to react for 3 h at a room temperature of 20° C. After the reaction was ended, the reaction product was extracted with bichloromethane and washed repeatedly with deionized water, and finally a crude product was obtained by rotary evaporation. The crude product was dried for 2 h in an oven at 60° C. to obtain a target product discoid liquid crystal small molecule BTA-1. The molecular formula of the discoid liquid crystal small molecule BTA-1 is as shown in FIG. 1 and FIGS. 5(a)-5(d).

The synthesized discoid liquid crystal small molecule BTA-1 was sampled and subjected to 1H-NMR analysis. The results are as shown in FIG. 1. It can be seen from the figure that there are peaks that correspond to the BTA-1 molecule, a ratio of peak integral areas is consistent with a hydrogen atom number ratio and there are no impurity peaks, indicating that the target molecule BTA-1 is successfully prepared, and the sample is pure and has no impurities.

### (2) Preparation of Polymer Composite Film

0.005 g of discoid liquid crystal small molecule BTA-1 was added into 2.25 mL of N, N-dimethylformamide solvent to be ultrasonically and uniformly mixed to obtain a discoid liquid crystal small molecule solution with a solid content of 0.22%, which was marked as solution A1. 0.5 g of PVDF powders were added into 2.25 mL of N, N-dimethylformamide solvent to be uniformly stirred to obtain a PVDF solution with a solid content of 22%, which was marked as solution A2. At a room temperature, 2.25 mL of solution A1 was added into 2.25 mL of solution A2 to be uniformly stirred to obtain a blended solution. The blended solution was cast onto a clean glass plate, and the glass plate was heated to 60° C. in an oven and dried for 3 h and then subjected to thermal treatment for 12 h at 110° C. to finally obtain a high-energy-storage high-density polymer film composite material containing 1 wt % of discoid liquid crystal molecule functional modifier.

Referring to the above-mentioned method, the amount of the discoid liquid crystal small molecule was changed to respectively prepare polymer film materials containing 1 wt %, 0.8 wt %, 0.6 wt %, 0.5 wt %, 0.4 wt %, 0.2 wt %, 0.1 wt %, 0.05 wt % and 0 (i.e., pure PVDF) discoid liquid crystal small molecule functional modifiers.

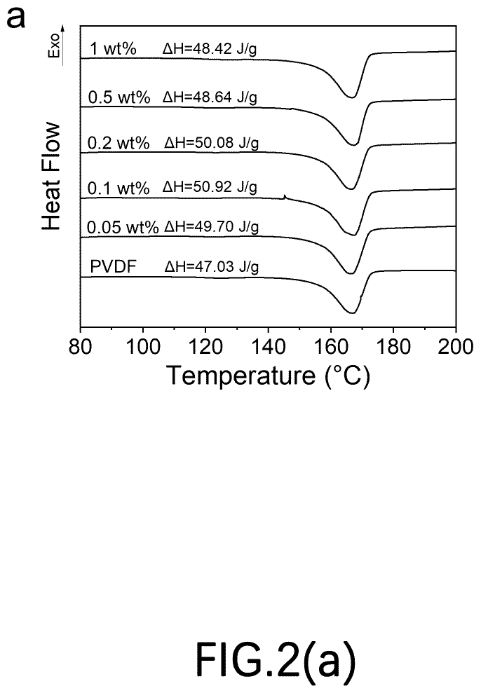

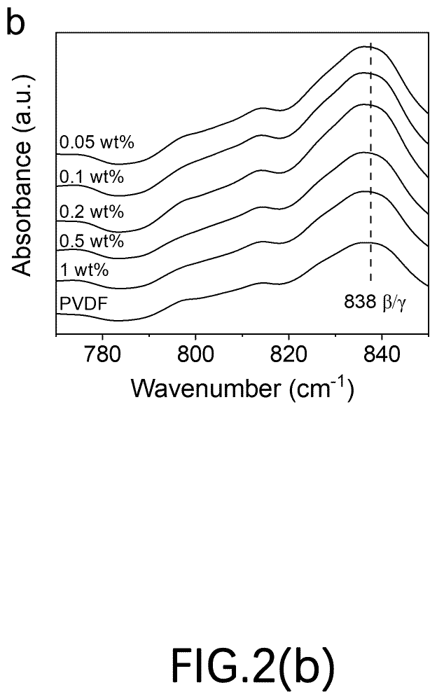

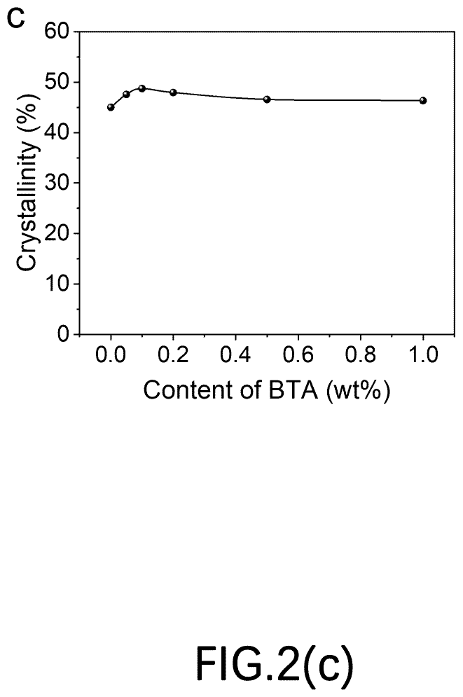

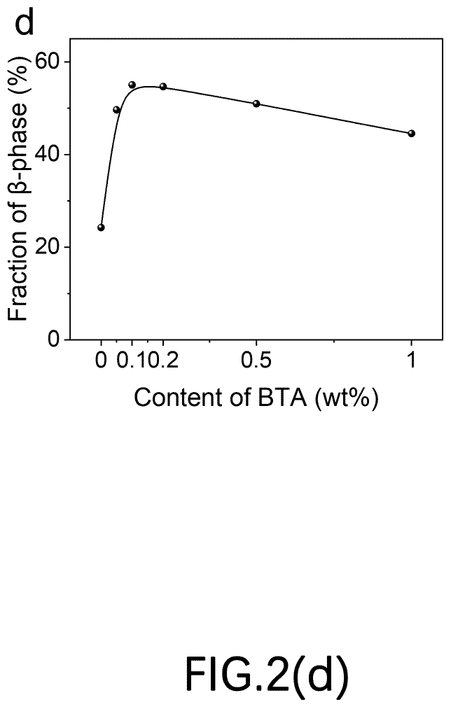

To sufficiently know the properties of these film materials, tests such as DSC, FTIR and XRD were respectively performed on the sample. The results are as shown in FIGS. 2(a)-2(d) respectively. The left upper graph of FIGS. 2(a)-2(d) is a DSC testing results graph of different film materials. It can be seen from the graph that the increase in BTA-1 filler can effectively improve the enthalpy of a melting peak, which is improved from 47.03 J/g for pure PVDF to 50.92 J/g. According to the crystallinity calculation equation: (Melting peak area-Cold crystallization peak area)×Theoretical enthalpy of 100%/100% crystalline material, the crystallinity of the film can be calculated, which is improved from 45% to 48.7%, and the specific results are as shown in a crystallinity line graph located at an upper right position of FIGS. 2(a)-2(d). The lower left graph of FIGS. 2(a)-2(d) is an FTIR test result graph of different film materials. It can be seen from the graph that the peaks at 839 cm⁻¹ corresponding to a β phase structure are increased compared with those of pure PVDF, indicating that the addition of BTA-1 can effectively improve the content of the β phase in the composite film. By quantitative calculation, the content of the β phase is increased from 24.2% to 55.0% to the maximum extent, and specifically shown in the β phase content in the right lower graph of FIGS. 2(a)-2(d).

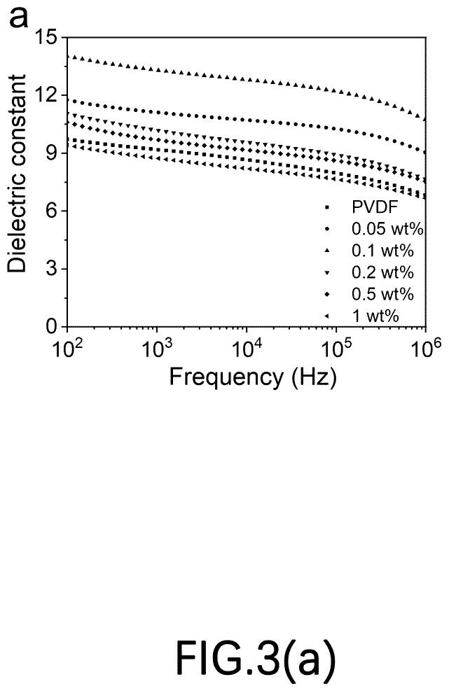

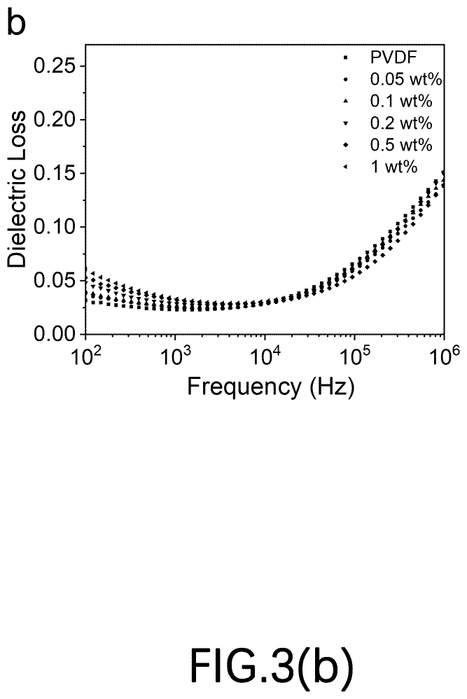

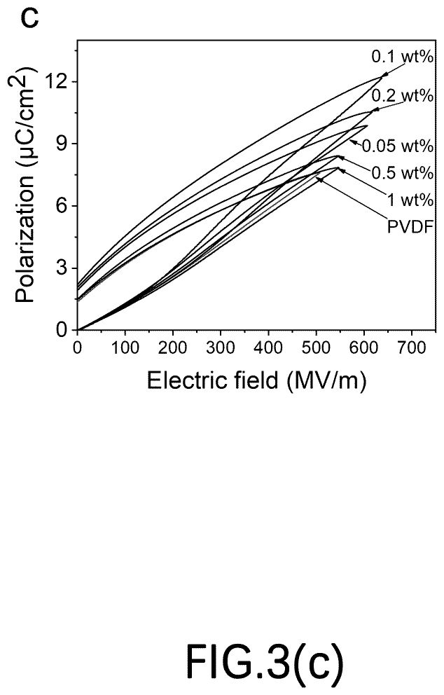

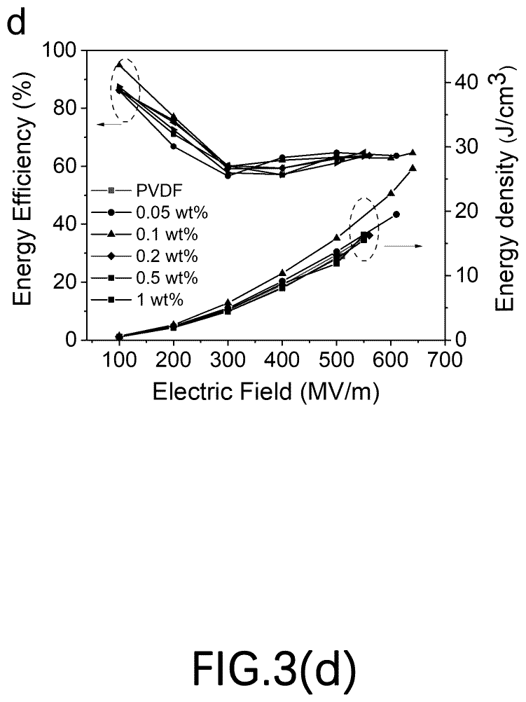

A series of polymer film materials prepared in example 1 were sampled and subjected to dielectric constant, dielectric loss and ferroelectric testing. Before electrical properties were measured, a circular gold electrode with a diameter of 1.2 mm, a thickness of 50 nm and an area of 0.04524 cm² was sputtered at the two sides of the film. The frequency-dependent dielectric constant and dielectric loss spectrum were measured using precise digital LCR instrument (E49080A, Agilent), with a frequency range of 20 Hz-2 MHz. The ferroelectric testing was conducted at a room temperature and 100 Hz using a ferroelectric testing system (Premier II, Radiant Technologies, Inc.). Relevant testing results are as shown in FIGS. 3(a)-3(d).

It can be seen from the upper left graph of FIGS. 3(a)-3(d) that the dielectric constant of the composite film with a content of 0.1 wt % is improved from 9.5 (pure PVDF) to 14; it can be seen from the upper right graph of FIGS. 3(a)-3(d) that the addition of BTA-1 makes the dielectric losses of the film materials reduced. The lower left graph of FIGS. 3(a)-3(d) is a D-E testing graph of different discoid liquid crystal small molecule contents under the maximum breakdown field strength. It can be seen from the figure that the ferroelectric properties of the composite thin film with a content of 0.1 wt % are maximally improved, and the maximum polarization intensity is increased from 8 μC/cm² to 12.1 μC/cm². The lower left picture of FIGS. 3(a)-3(d) shows energy storage density and energy storage efficiency of different composite film contents. It can be seen from the broken line graph that the energy storage density is maximally improved to 26. 7 J/cm³ under the condition that the energy storage efficiency is not reduced. The above-mentioned dielectric and ferroelectric graphs indicate that BTA-1 can greatly improve the electrical properties of the composite film under the condition of extremely low addition amount (0.1 wt %).

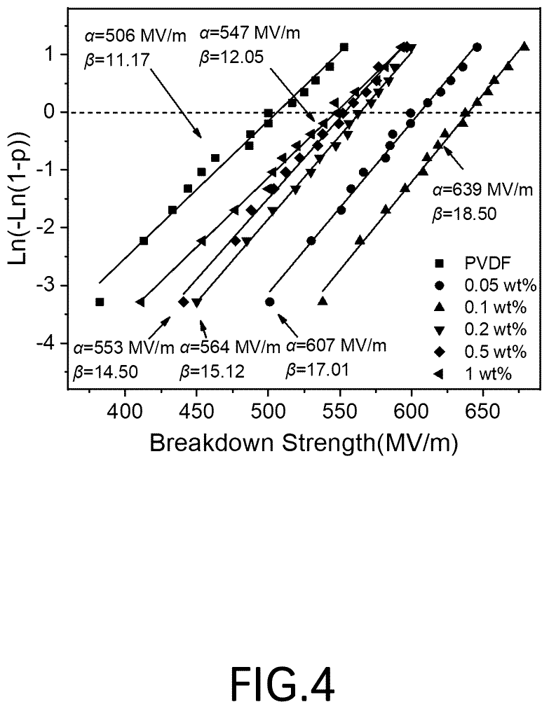

The breakdown strength testing results of a series of polymer film materials prepared in example 1 are as shown in FIG. 4. Breakdown testing was conducted using dielectric voltage withstand testing system (Beijing Institute of Mechanical and Electrical Engineering Overvoltage Technology). 15 to 20 points were measured for each sample, and a breakdown field strength Weber distribution graph was obtained through linear fitting calculation using a dual-parameter Weibull statistical distribution. It can be seen from the figure that the breakdown field strength of the film is improved from 506 MV/m (pure PVDF) to 639 MV/m, indicating that the addition of BTA-1 can effectively improve the breakdown performance of the composite film.

### Example 2

### (1) Synthesis of Discoid Liquid Crystal Small Molecule BTA-2

3.76 mmol of 1,3,5-benzoyl chloride and 12.4 mmol of ω-amino acid were added into 30 mL of trichloromethane in a nitrogen atmosphere under the conditions of no water and oxygen at 0° C. to be uniformly mixed, and then the obtained mixture was subjected to a thermal insulation reaction for 30 min at 0° C. and subsequently continued to react for 3 h at a room temperature of 20° C. After the reaction was ended, the reaction product was extracted with bichloromethane and washed repeatedly with deionized water, and finally a crude product was obtained by rotary evaporation. The crude product was dried for 2 h in an oven at 60° C. to obtain a target product discoid liquid crystal small molecule BTA-2. The molecular formula of the discoid liquid crystal small molecule BTA-2 is as shown in FIGS. 5(a)-5(d).

### (2) Preparation of Polymer Composite Film

50 g of polypropylene pellets was weighed at a time and spread in a culture dish, so as to ensure that the culture dish was covered with at least two layers of polypropylene pellets, and subsequently the culture dish was transferred to a heating stage to be heated for 40 min at a temperature of no more than the melting temperature of the polymer. 0.05 g of discoid liquid crystal small molecule BTA-2 prepared in step (1) and 10 mL of ethanol were uniformly mixed to obtain a discoid liquid crystal small molecule solution with a concentration of 5 g/L. The discoid liquid crystal small molecule solution was uniformly sprayed onto the surfaces of the polypropylene pellets and then dried in vacuum for 2 h at 90° C.

The cooling water in an extruder was opened, the temperatures of four-stage heating regions were adjusted and respectively set as 130° C., 140° C., 200° C. and 190° C., and the temperatures were maintained for 5-30 min after being raised to the set temperatures. Screw rotation was opened, the treated polymer composite pellets were added for extrusion, and the materials were always kept in the flow channel to ensure the extrusion pressure unchanged. When the melt flew out from the head of the extruder, a puller is started, and a distance between the puller and the head of the extruder was controlled to about 30 cm to ensure the cooling during the pulling. When the melt was pulled and adhered to a roller, the pulling speed gradually increased but was no more than 30 r/min, thereby enabling the polymer composite film material to conduct continuous large-scale melt extrusion.

### Example 3

### (1) Synthesis of Discoid Liquid Crystal Small Molecule BTA-3

3.76 mmol of 1,3,5-benzoyl chloride and 10.9 mmol of n-heptamine were added into 30 mL of trichloromethane in a nitrogen atmosphere under the conditions of no water and oxygen at 0° C. to be uniformly mixed, and then the obtained mixture was subjected to a thermal insulation reaction for 30 min at 0° C. and subsequently continued to react for 3 h at a room temperature of 20° C. After the reaction was ended, the reaction product was extracted with bichloromethane and washed repeatedly with deionized water, and finally a crude product was obtained by rotary evaporation. The crude product was dried for 2 h in an oven at 60° C. to obtain target product discoid liquid crystal small molecule BAT-3. The molecular formula of the discoid liquid crystal small molecule BAT-3 is as shown in FIGS. 5(a)-5(d).

### (2) Preparation of Polymer Composite Film

50 g of polypropylene pellets was weighed at a time and spread in a culture dish, wherein the culture dish was covered with at least two layers of polypropylene pellets, and subsequently the culture dish was transferred to a heating stage to be heated for 40 min at a temperature of no more than the melting temperature of the polymer. 0.05 g of discoid liquid crystal small molecule BTA-3 prepared in step (1) and 10 mL of ethanol were uniformly mixed to obtain a discoid liquid crystal small molecule solution with a concentration of 5 g/L. The discoid liquid crystal small molecule solution was uniformly sprayed onto the surfaces of the polypropylene pellets and then dried in vacuum for 2 h at 90° C.

The cooling water in an extruder was opened, the temperatures of four-stage heating regions were adjusted and respectively set as 130° C., 140° C., 200° C. and 190° C., and the temperatures were maintained for 5-30 min after being raised to the set temperatures. Screw rotation was opened, the treated polymer composite pellets were added for extrusion, and the materials were always kept in the flow channel to ensure the extrusion pressure unchanged. When the melt flew out from the head of the extruder, a puller is started, and a distance between the puller and the head of the extruder was controlled to about 35 cm to ensure the cooling during the pulling. When the melt was pulled and adhered to a roller, the pulling speed gradually increased but was no more than 30 r/min, thereby enabling the polymer composite film material to conduct continuous large-scale melt extrusion.

### Example 4

### (1) Synthesis of Discoid Liquid Crystal Small Molecule BTA-4

3.76 mmol of 1,3,5-benzoyl chloride and 12.8 mmol of ω-amino acid were added into 30 mL of trichloromethane in a nitrogen atmosphere under the conditions of no water and oxygen at 0° C. to be uniformly mixed, and then the obtained mixture was subjected to a thermal insulation reaction for 30 min at 0° C. and subsequently continued to react for 3 h at a room temperature of 20° C. After the reaction was ended, the reaction product was extracted with bichloromethane and washed repeatedly with deionized water, and finally a crude product was obtained by rotary evaporation. The crude product was dried for 2 h in an oven at 60° C. to obtain a target product discoid liquid crystal small molecule BTA-4. The molecular formula of the discoid liquid crystal small molecule BTA-4 is as shown in FIGS. 5(a)-5(d).

### (2) Preparation of Polymer Composite Film

0.004 g of discoid liquid crystal small molecule BTA-4 was added into 2.25 mL of N, N-dimethylformamide solvent to undergo ultrasonic treatment so as to be uniformly mixed, such that a discoid liquid crystal small molecule solution with a solid content of 0.18% was obtained, which was marked as solution A1. 0.5 g of P(VDF-TrFE) powders were added into 2.25 mL of N, N-dimethylformamide solvent to be uniformly stirred to obtain a P(VDF-TrFE) solution with a solid content of 22%, which was marked as solution A2. 2.25 mL of solution A1 was added into 2.25 mL of solution A2 at a room temperature to be uniformly stirred to obtain a blended solution. The blended solution was cast onto a clean glass plate, and then the glass plate was heated to 60° C. in an oven and dried for 3 h, then subjected to thermal treatment for 12 h at 110° C. to finally obtain a high-energy-storage high-density polymer film composite material containing 0.8 wt % discoid liquid crystal molecule functional modifier.

The inventor conducted tests under different parameter matching conditions in the process parameter range disclosed in the technical solution and obtained good effects.

What is claimed is:

1. A discoid liquid crystal molecule functional modifier, the functional modifier having the following molecule structure: 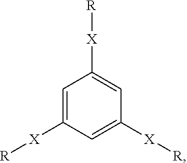
wherein X is selected from any one of —CONH—, —NHCO—, —O—, —COO—, —OOC—, —C═O— and —OCO—, and R is selected from any one of —(CH₂)ₓCH₃, —(CH₂)ₓCOOH, —(CH₂)ₓOH, —(CH₂)ₓNH₂, —(CH₂)ₓCONH(CH₂)_{Y}CH₃, —(CH₂)ₓNHCO(CH₂)_{Y}CH₃, —(CH₂)ₓCOO(CH₂)_{Y}CH₃, —(CH₂)ₓOOC(CH₂)_{Y}CH₃, —(CH₂)ₓCO(CH₂)_{Y}CH₃, —(CH₂)ₓO(CH₂)_{Y}CH₃ or flexible side chains of the aforementioned groups substituted by a halogen atom, an amino group, a carboxyl group, a hydroxyl group and a sulfo group at any positions, and the values of X and Y are positive integers within 18 (inclusive).

2. The discoid liquid crystal molecule functional modifier according to claim 1, wherein X is specifically —CONH— or —NHCO—, and R is specifically —(CH₂)₅CH₃, —(CH₂)₅COOH, —(CH₂)₆CH₃ or —(CH₂)₆NH₂.

3. A method for preparing the discoid liquid crystal molecule functional modifier according to claim 1 or 2, wherein the method comprises the following steps: reacting by using a trimesoyl chloride monomer and a flexible carbon chain compound with an amino group as raw materials under the conditions of no water, no oxygen and no more than 5° C., separating and purifying to obtain the discoid liquid crystal molecule functional modifier.

4. The method according to claim 3, wherein the flexible carbon chain compound with the amino group is specifically organic amine or amino acid, which comprises n-hexamine, n-heptamine, n-octylamine and ω-amino acid; a molar ratio of the trimesoyl chloride monomer to the flexible carbon chain compound with the amino group required by reaction is 1:2.2-3.8.

5. The method according to claim 3, wherein the specific process of the reaction is as follows: dissolving the trimesoyl chloride monomer and the flexible carbon chain compound with the amino group into an organic solvent under the conditions of no water, no oxygen and no more than 5° C. to be uniformly mixed, then performing a thermal insulation reaction followed by heating to room temperature to continue the reaction, and then subjecting the reaction product to organic solvent extraction, water washing, column chromatography separation or recrystallization, rotary evaporation and drying, so as to obtain a target product.

6. Use of the discoid liquid crystal molecule functional modifier according to claim 1 or 2 in adjusting and controlling the crystalline structure and performance of a polymer film material.

7. The use according to claim 6, wherein the use method of the discoid liquid crystal molecule functional modifier is specifically as follows: (a) respectively dissolving the discoid liquid crystal molecule functional modifier and a polymer into a solvent, then uniformly mixing two solutions and forming a film; or (b) heating and melting the polymer, then adding the discoid liquid crystal molecule functional modifier, and performing extrusion filming.

8. The use according to claim 6, wherein the polymer is selected from at least one of polyvinylidene fluoride, polyvinylidene difluoride-hexafluoropropene, a polyvinylidene fluoride-trifluoroethylene copolymer, a polyvinylidene fluoride-chlorotrifluoroethylene copolymer, an ethylene-tetrafluoroethylene copolymer, an fluorinated ethylene propylene copolymer, polychlorotrifluoroethylene, a copolymer of perfluoropropyl perfluorovinyl ether and polytetrafluoroethylene, polyvinyl chloride, polyhexamethylene adipate, polyamide, polyethylene terephthalate, polymethyl methacrylate, polypropylene and polyphenylene sulfide; the mass fraction of the discoid liquid crystal molecule functional modifier in the polymer film material is 0.0001%-10%, the thickness of the film material is 1 μm-100 μm; in the method (a), the solvent for dissolving the discoid liquid crystal molecule functional modifier and the polymer is specifically a non-protonic solvent comprising N-methylpyrrolidone, N, N′-dimethylacetamide, N, N′-dimethylformamide and ethanol.

9. The use according to claim 6, wherein in the method (a), the solid contents of the discoid liquid crystal molecule functional modifier solution and the polymer solution which are obtained by dissolution are 0.001 wt %-50 wt % and 1 wt %-30 wt % respectively, a volume ratio of the two solutions during the mixing is 1:20-20:1, the film formation method is selected from at least one of tape casting, spin coating and pulling methods; in the method (b), the polymer is first heated and melted, and then a discoid liquid crystal molecule functional modifier solution with a solid content of 0.001 wt %-50 wt % is prepared by referring to the method (a), subsequently the prepared discoid liquid crystal molecule functional modifier solution is uniformly sprayed to the surface of the polymer melt, and the obtained mixed material is dried in vacuum and then subjected to extrusion filming in an extruder.

10. The use of according to claim 6, wherein in the method (b), the vacuum drying temperature of the mixed material is 40° C.-100° C., the mixed material is heated in the extruder in four stages, the heating temperatures of the four stages are 120° C.-140° C., 130° C.-150° C., 180° C.-220° C. and 170° C.-210° C. in sequence, and the extruded material is pulled by a puller at the pulling speed of no more than 30 r/min, wherein a distance between the puller and the extruder head is 15-45 cm.
